(19) United States
(12) Patent Application Publication
(10) Pub. No.: US 2010/0222760 A1
(43) Pub. Date: Sep. 2, 2010

(54) TRAINING PANTS WITH AGE APPROPRIATE GRAPHICS

(75) Inventors: Matthew J. Rinaldi, Englishtown, NJ (US); M. Reid Macfarlan, Allentown, NJ (US); Charles F. Schroer, JR., Camden, AR (US)

Correspondence Address: DICKSTEIN SHAPIRO LLP, 1633 Broadway, NEW YORK, NY, 10019, US

(73) Assignee: ARQUEST, INC., Cranbury, NJ (US)

(21) Appl. No.: 12/395,451

(22) Filed: Feb. 27, 2009

(51) Int. Cl. A61F 13/49

(52) U.S. Cl. 604/385.01

(57) ABSTRACT

A series of disposable absorbent articles designed to fit wearers at different stages of development including a first absorbent article configured to fit a wearer in a first stage of development and a second absorbent article configured to fit a wearer in a second stage of development different than the first stage. The first and second absorbent articles have a common character graphic thereon, the graphic depicted differently in the first and second absorbent articles.

### FIELD OF THE INVENTION

The present invention relates generally to absorbent articles, and more particularly to a series of disposable absorbent articles having common character graphics.

### BACKGROUND OF THE INVENTION

Disposable absorbent articles such as diapers and training pants are designed to absorb and contain bodily waste to prevent soiling of the body and clothing. These articles are available in a variety of different sizes, styles and absorbencies to fit a variety of wearers ranging from newborns to active toddlers. Absorbent articles often include attractive pattern and color combinations, graphics, or other indicia for attracting and identifying with the wearer.

As a wearer progresses through to potty training, he or she may go through several types of disposable absorbent articles. Over time, the wearer can develop a connection with a certain character or graphic on the absorbent article. Once this connection has been formed, the wearer may not wish to bid farewell to the character or graphic they have grown accustom to. As a result, the transition between absorbent articles may become increasingly difficult. Ultimately, the wearer's potty training may be inhibited.

### SUMMARY OF THE INVENTION

Accordingly, there is a need in the art for a series of absorbent articles having a common character graphic that wearers can identify with through their various stages of development and potty training. Providing such a series of articles with a common character graphic addresses the above-noted attachment issues of a wearer. The present invention allows a wearer to make a connection with a certain character graphic, or series of graphics, and to maintain this connection as the wearer progresses through the series of disposable absorbent articles for the duration of the potty training process.

In accordance with the present invention, as wearers progress from one absorbent article to another, they will continue seeing the common character graphic they have grown accustom to and will identify with the changed appearance of the character graphic as signifying the wearer's own potty training progression. Including the common character graphic on different absorbent articles in a series puts the wearer at ease during potty training and facilitates a smoother transition between different stages of absorbent articles. The wearer can associate his own succession through the different absorbent articles in the series with the increased growth and development of the common character graphics. Furthermore, as the wearer grows and moves into larger absorbent articles, his desire to stay connected to the character graphic will drive his caretaker to purchase the next size absorbent article in the series resulting in brand loyalty. The caretaker will also more easily identify which absorbent article type is most appropriate for their child through single identification of the common character graphic.

The present invention provides a series of disposable absorbent articles designed to fit wearers at different stages of development. The series includes a first absorbent article configured to fit a wearer in a first stage of development and a second absorbent article configured to fit a wearer in a second stage of development, which is different than the first stage. The first and second absorbent articles have a common character graphic thereon, except that the character graphic is depicted differently in the first and second absorbent articles.

The present invention also provides a method of concurrently providing a series of disposable absorbent articles to consumers, in which each absorbent article for a different stage of development displays a common character graphic. The method includes providing a first absorbent article targeted to fit wearers of a first stage of development; providing a second absorbent article targeted to fit wearers of a second stage of development different than the first stage of development; displaying a common character graphic of a general theme on the first and second absorbent articles; and depicting the common character graphic differently on the first and second absorbent articles.

These and further features and advantages of the present invention will become more apparent from the following description when taken in connection with the accompanying drawings which show, for purposes of illustration only, an embodiment in accordance with the present invention.

### BRIEF DESCRIPTION OF THE DRAWINGS

The same or corresponding components in the following drawings are denoted by the same reference numerals.

FIG. 1 is a perspective view of a series of absorbent articles according to an embodiment of the present invention.

### DETAILED DESCRIPTION OF PREFERRED EMBODIMENTS OF THE INVENTION

As used herein, the general term “graphic” is used to mean any design, pattern, figure or the like that is or becomes visible on an absorbent article, and specifically includes pictorial images that consist of one or more pictures and combinations thereof.

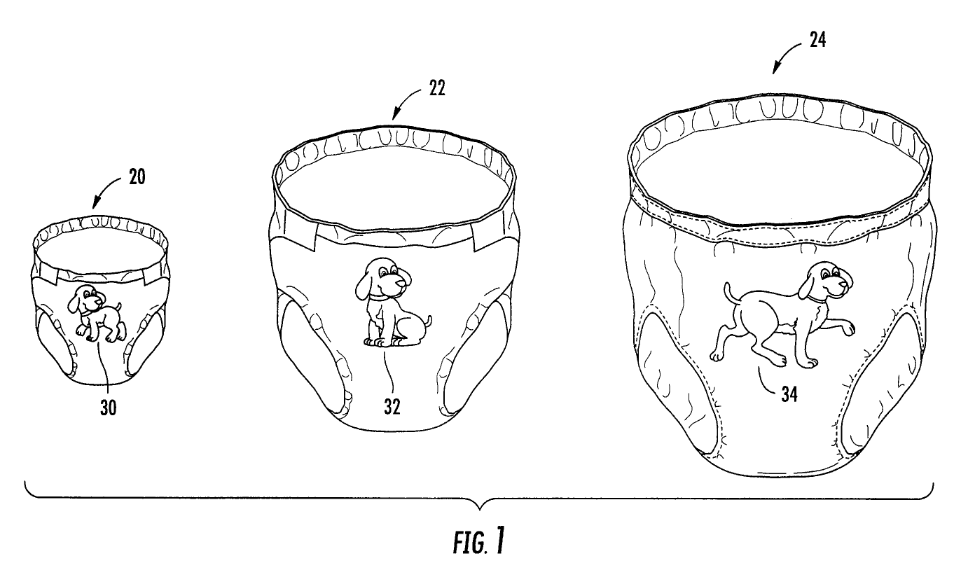

FIG. 1 and the embodiment used to describe the principles of the present invention is by way of illustration only and should not be construed in any way to limit the scope of the invention. Discussion of well-known absorbent article features and components has been omitted insomuch as such details are not necessary to obtain a complete understanding of the present invention and are within the skill of a person of ordinary skill in the relevant art.

The principles of the present invention can be incorporated into a series of two or more absorbent articles. Examples of suitable absorbent articles include, but are not limited to, diapers and training pants. The absorbent articles each have a common character graphic identifiable thereon which varies in some manner so as to distinguish each article from each other article in the series. For example, the series of absorbent articles display an animated dog as a character common to each article in the series (e.g., a cartoon dog of a children's television series or the like), whereby on each different absorbent article in the series, the animated dog is depicted at a different age and/or engaging in an activity appropriate for the intended age range for that absorbent article.

Referring to FIG. 1, absorbent articles 20, 22 and 24 include character graphics 30, 32 and 34, respectively. One exemplary arrangement of absorbent articles 20, 22 and 24 can include article 20 being a diaper for immobile infants; article 22 being a diaper for mobile infants and crawling toddlers; and article 24 being a training pant for walking toddlers. In accordance with alternative arrangements, article 20 can be a diaper and articles 22 and 24 can be different stage training pants for later stages of infant or toddler development. Absorbent articles 20, 22 and 24 can comprise any combination of absorbent articles. For example, articles 20, 22 and 24 can comprise all diapers or training pants, wherein each article 20, 22 and 24 has some characteristic or trait which is different for each other article in the series.

Character graphics 30, 32 and 34 depict a character common to absorbent article 20, 22 and 24, respectively. While each character graphic 30, 32 and 34 has a common characteristic, each graphic is preferably depicted differently. Common character graphics can include portraying a character, common to each article, that is participating in a different activity or showing the physiological growth of the character from one graphic to the next across the series of absorbent articles.

FIG. 1 illustrates one arrangement of how a common character can be depicted differently over a series of absorbent articles 20, 22 and 24. For purposes of illustration, the absorbent article series of FIG. 1 includes three absorbent articles 20, 22 and 24, however, the series can include as few as two absorbent articles, or as many different absorbent articles as necessary to address the needs of a child during various stages of toilet training. The common character graphic on absorbent articles 20, 22 and 24 is an animated dog. Character graphics 30, 32 and 34 each include the animated dog, yet the dog is depicted differently on each article 20, 22 and 24. Character graphic 30 depicts the animated dog as a newborn puppy or in a newborn puppy stage of development; character graphic 32 depicts the animated dog in a later stage of development, i.e., in a toddler puppy stage of development; and character graphic 34 depicts the animated dog in a full-grown stage of development (e.g., as full-sized dog).

Character graphics 30, 32, and 34 can be used to provide a story line involving a common character. For example, in the embodiment shown in FIG. 1, the animated dog is depicted as growing older and more mature as the wearer advances from absorbent article 20 to absorbent article 22 to absorbent article 24. Suitable character graphics can include animals, people, inanimate objects, natural phenomena, cartoon characters, or the like that may be provided with human features such as arms, legs, facial features or the like. For purposes of enhanced toilet training, it may be desirable for the character graphic to be familiar to the child, such as an identifiable cartoon character like Mickey Mouse®. The character graphics are preferably a type that the child can relate to, examples of which could include animals, toys, licensed characters, or the like. Character graphics can be made more personable and friendly to the child by including human-like features, human-like expressions, apparel, abilities, or the like. By way of illustration, an animal character graphic can be shown smiling, wearing clothing, playing sports, playing with toys, or the like. In alternative embodiments, the character graphic can desirably be created to project an appearance that could be described as friendly, positive, non-intimidating, silly, independent, inspirational, active, expressive, dauntless and/or persevering.

Ideally, the character graphic is suitable for children's absorbent articles. To that end, the character graphics can be associated with popular characters in the media, advertising, or well known in a particular culture. Ideally, they are characters that the child or caregiver care about and want to identify with. Ideally, the child can imagine himself or herself taking the place of the character.

Absorbent articles 20, 22 and 24 are indicated as having different absorbent capacities to reflect needs of wearers as they progress through potty training. Thus, in one embodiment, as the wearer progresses from early stage absorbent article 20 to middle stage absorbent article 22, the depiction of the character graphic progresses from the animated dog as a newborn puppy 30 to an adolescent dog 32. Accordingly, a wearer of this absorbent article series can associate his progression from absorbent article 20 to absorbent article 22 with a change from character graphics 30 and 32. In this particular example, the animated dog gets older as the wearer progresses into the next level of absorbent article. However, the character graphic can alternatively be shown getting younger, or staying the same age, or, more simply, just participating in different activities. In different embodiments of the present invention, each level of the absorbent article can be based on the wearer's age, size or the like.

Including a common character graphic variously depicted over a series of absorbent articles benefits both the wearer and the caretaker. In particular, as a wearer becomes accustom to and builds an attachment with a particular character, he may be hesitant to switch to another absorbent article having a different character as his needs change over time for failure to “let go” of his bond to that the character. Including a common character graphic on different absorbent articles in a series can assist in putting the wearer at ease and can facilitate a smoother transition between the different stages of absorbent articles. Furthermore, with the present invention, the wearer can associate his own succession through absorbent articles with the increased growth and development of character graphics 30, 32 and 34. This correlation among character graphic and absorbent article not only comforts the wearer as he naturally progresses between absorbent articles 20, 22 and 24, but also motivates the wearer to ascend to the next character graphic in the absorbent article series. In other words, the wearer will come to associate his progression between absorbent articles with the depiction of the common character graphics thereon.

Associating character graphics with absorbent articles also provides benefits to the caretakers. Since the caretaker is typically responsible for selecting and purchasing absorbent articles for their child, the caretaker will be able to more easily identify which absorbent article type is most appropriate for their child through single identification of the common character graphic. For example, a caretaker looking to purchase an early stage diaper for her immobile infant will take notice of the common character as a means of identifying the proper absorbent article in the series. Similar to how absorbent articles can be identified by their size, design and absorbent capacity, an absorbent article series in accordance with the present invention can further be identified by the depiction of a character graphic on the respective absorbent articles or their packaging, making up the series itself. For example, a specific depiction of a character graphic can be associated with a particular size and/or absorbent capacity. Thus, the caregiver can identify a specific absorbent article's size and absorbency simply by referring to the character graphic depicted on the article or its associated packaging and purchasing materials. This makes identifying a proper absorbent article for their child easier. Furthermore, as the wearer progresses into different types of absorbent articles in the series, the caregiver can easily identify the appropriate article for her child, as well as enjoy the excitement and surprise of a familiar, yet differently depicted, character graphic on the newly selected absorbent article. Accordingly, both caregiver and wearer can share in the experience of potty training by associating the wearer's development through the series of absorbent articles with different depictions of a common character graphic embodied on the articles.

In carrying out the method of the invention, a series of absorbent articles 20 and 22 are provided to consumers, each absorbent article has at least one characteristic distinct from each other absorbent article in the series. A common character graphic 30, 32 is displayed on each respective absorbent article 20, 22. For purposes of illustration, a method in accordance with the present invention is shown utilizing only absorbent articles 20 and 22. It is to be understood that the disclosed method is not limited to two absorbent articles and can be implemented with any suitable number of absorbent articles. For example, the method can employ three absorbent articles 20, 22 and 24 as shown in FIG. 1, but, in the alternative, can utilize any number of absorbent articles consistent with the purposes of the invention.

The method is achieved by providing a first absorbent article targeted to fit wearers of a first stage of development. The first absorbent article could resemble absorbent article 20 of FIG. 1. Next, a second absorbent article targeted to fit wearers of a second stage of development different than the first stage of development is provided. The second absorbent article could resemble absorbent article 22 of FIG. 1. A common character graphic of a general theme is displayed on both the first and second absorbent articles, with the common character graphic being depicted differently on the first and second absorbent articles, respectively. The first and second graphics could resemble common character graphics 30 and 32 of FIG. 1.

The method of the invention is applicable to absorbent articles including diapers and training pants. As discussed with reference to FIG. 1, the difference between the common character graphic 30 of the first article 20 and the common character graphic 32 of the second article 22 can be the stage of development of the character depicted therein. The difference between the first and second common character graphics 30 and 32 can be anything that distinguishes the graphics from one another while maintaining a similar or substantially similar image.

The character graphics can be located on the exterior surface of the absorbent article. Alternatively, however, the character graphics can be located on the interior surface of the absorbent article or between layers of a multilayer absorbent article, provided the graphics remain visible from the exterior of the article.

It will be appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

We claim:

1. A plurality of disposable absorbent articles comprising:
a first absorbent article configured to fit a wearer in a first stage of development; and
a second absorbent article configured to fit a wearer in a second stage of development, which is different than the first stage of development,
wherein the first and second absorbent articles have a common character graphic thereon, which is depicted differently on the first and second absorbent articles.

2. The plurality of disposable absorbent articles of claim 1, wherein the depiction of the common character graphic on the first absorbent article is associated with the first stage of development and the depiction of the common character graphic on the second absorbent article is associated with the second stage of development.

3. The plurality of disposable absorbent articles of claim 1, wherein the first and second stages of development are different sizes.

4. The plurality of disposable absorbent articles of claim 3, wherein the second size is larger than the first size.

5. The plurality of disposable absorbent articles of claim 1, wherein the first and second stages of development are different age ranges.

6. The plurality of disposable absorbent articles of claim 5, wherein the second age range is older than the first age range.

7. The plurality of disposable absorbent articles of claim 1, wherein the common character graphic is part of a general theme and each different depiction is illustrated as performing a different activity.

8. The plurality of disposable absorbent articles of claim 1, wherein the first absorbent article has a first absorbency characteristic; and
the second absorbent article has a second absorbency characteristic different than the first absorbency characteristic.

9. The plurality of disposable absorbent articles of claim 8, wherein the second absorbency characteristic is more absorbent than the first absorbency characteristic.

10. The plurality of disposable absorbent articles of claim 1, wherein the character is depicted differently based on the stage of development that the absorbent article is targeted to serve.

11. The plurality of disposable absorbent articles of claim 1, further comprising a third absorbent article configured to fit a wearer in a third stage of development, wherein the first, second and third absorbent articles have a common character graphic thereon, which is depicted differently on each of the absorbent articles.

12. A set of disposable absorbent articles designed to fit wearers at different stages of development comprising:
a plurality of first absorbent articles; and
a plurality of second absorbent articles;
wherein the plurality of first absorbent articles and the plurality of second absorbent articles have a common character graphic thereon, which is depicted differently on the plurality of first absorbent articles and the plurality of second absorbent articles.

13. The set of disposable absorbent articles of claim 12, wherein the depiction of the common character graphic on the plurality of first absorbent article is associated with a first stage of development and the depiction of the common character graphic on the plurality of second absorbent article is associated with a second stage of development.

14. The set of disposable absorbent articles of claim 12, wherein each of the plurality of first absorbent articles are configured to fit a wearer of a first size and each of the plurality of second absorbent articles are configured to fit a wearer of a second size.

15. The set of disposable absorbent articles of claim 14, wherein the second size range is larger than the first size range.

16. The set of disposable absorbent articles of claim 14, wherein the character is depicted differently based on the size of the wearer that the absorbent article is targeted to serve.

17. The set of disposable absorbent articles of claim 12, wherein each of the plurality of first absorbent articles are configured to fit a wearer in a first age range and each of the plurality of second absorbent articles are configured to fit a wearer in a second age range.

18. The set of disposable absorbent articles of claim 15, wherein the second age range is older than the first age range.

19. The set of disposable absorbent articles of claim 15, wherein the character is depicted differently based on the age range of the wearer that the absorbent article is targeted to serve.

20. The set of disposable absorbent articles of claim 12, wherein the common character graphic is part of a general theme and each different depiction is illustrated as performing a different activity.

21. The set of disposable absorbent articles of claim 12, wherein the plurality of first absorbent articles has a first absorbency characteristic and the plurality of second absorbent article has a second absorbency characteristic that is different from the first absorbency characteristic.

22. The set of disposable absorbent articles of claim 21, wherein the second absorbency characteristic is more absorbent than the first absorbency characteristic

23. The set of disposable absorbent articles of claim 22, wherein the character is depicted differently based on the absorbency of the absorbent article.

24. The set of disposable absorbent articles of claim 12, further comprising a plurality of third absorbent articles configured to fit a wearer in a third stage of development, wherein the first, second and third pluralities of absorbent articles have a common character graphic thereon, which is depicted differently on each of the plurality of absorbent articles.

25. A method of concurrently providing a series of disposable absorbent articles to consumers, with each absorbent articles in the series provided for a different stage of development, the method comprising:
providing a first absorbent article targeted to fit wearers in a first stage of development;
providing a second absorbent article targeted to fit wearers in a second stage of development different than the first stage of development;
displaying a common character graphic of a general theme on the first and second absorbent articles; and
depicting the common character graphic differently on the first and second absorbent articles, respectively.

26. The method of claim 25, wherein the depicting step further comprises:
depicting the common character graphic on the first absorbent article to be associated with the first stage of development; and
depicting the common character graphic on the second absorbent article to be associated with the second stage of development.
